(19) United States
(12) Patent Application Publication
(10) Pub. No.: US 2026/0219343 A1
(43) Pub. Date: Jul. 30, 2026

(54) METHOD AND SYSTEM FOR MOTION CORRECTION OF MAGNETIC RESONANCE IMAGES

(71) Applicant: GE Precision Healthcare LLC, Waukesha, WI (US)

(72) Inventors: Gaspar Delso, Barcelona (ES); Martin Andreas Janich, Munich (DE)

(73) Assignee: GE Precision Healthcare LLC, Waukesha, WI (US)

(21) Appl. No.: 19/039,317

(22) Filed: Jan. 28, 2025

(51) Int. Cl. G01R 33/56; G01R 33/50; G01R 33/565

(52) U.S. Cl. CPC G01R 33/5608; G01R 33/50; G01R 33/5601; G01R 33/56509

(57) ABSTRACT

A method of processing magnetic resonance (MR) images includes obtaining a first series of MR image frames of a patient's anatomy over a first time period and generating a first quantitative parameter map based on the first series of MR image frames, then obtaining a second series of MR image frames of the patient's anatomy over a second time period, and generating a second quantitative parameter map based on the second series of MR image frames. One MR image frame is selected from the first series of MR image frames as a first representative image and one MR image frame is selected from the second series of MR image frames as a second representative image. A misalignment valuation is then calculated based on a comparison of the first representative image to the second representative image. The misalignment valuation is then used to align the first and second quantitative maps.

### BACKGROUND

The present disclosure generally relates to magnetic resonance imaging systems, and specifically to systems and methods for motion correction for aligning two quantitative parameter maps based on different magnetic resonance series taken at disparate times.

MR imaging (MRI) has proven useful in diagnosis of many diseases. MRI provides detailed images of soft tissues, abnormal tissues such as tumors, and other structures, which cannot be readily imaged by other imaging modalities, such as computed tomography (CT). Further, MRI operates without exposing patients to ionizing radiation experienced in modalities such as CT and x-rays. MRI is often used to obtain internal physiological information about a patient, including for brain imaging, thoracic imaging, spine imaging, cardiac imaging, and imaging other sections or tissues within a patient's body (anywhere on the patient). Additionally, many MRI systems enable multi-anatomy or whole body imaging where a substantial portion of the patient's body is imaged, which is typically performed by separately imaging several portions of the patient's body and then stitching the images together to generate one continuous image.

MRI uses the nuclear magnetic resonance (“NMR”) phenomenon to produce images. When a substance such as human tissue is subjected to a uniform magnetic field, such as the so-called main magnetic field (polarizing field B₀) generated by an MRI system, the individual magnetic moments of the nuclei in the tissue attempt to align with this B₀ field, but precess about it in random order at their characteristic Larmor frequency. If the substance, or tissue, is subjected to a magnetic field (excitation field B1) which is in the x-y plane and which is near the Larmor frequency, the net aligned moment, or “longitudinal magnetization”, Mz, may be rotated, or “tipped”, into the x-y plane to produce a net transverse magnetic moment Mt. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradients, sometimes referred to as readout gradients, vary according to the particular localization method being used. The resulting set of received signals are digitized and processed to reconstruct the image using reconstruction techniques.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the disclosure, a method of processing magnetic resonance (MR) images includes obtaining a first series of MR image frames of a patient's anatomy over a first time period and generating a first quantitative parameter map based on the first series of MR image frames, then obtaining a second series of MR image frames of the patient's anatomy over a second time period, wherein the first time period does not overlap with the second time period, and generating a second quantitative parameter map based on the second series of MR image frames. At least one MR image frame is selected from the first series of MR image frames as a first representative image and at least one MR image frame is selected from the second series of MR image frames as a second representative image. A misalignment valuation is then calculated based on a comparison of the first representative image to the second representative image. The misalignment valuation is then used to align the first and second quantitative maps.

In one embodiment, the method further includes comparing the first quantitative parameter map and the second quantitative map based on the misalignment valuation to generate a comparison map.

In one embodiment, the first representative image is a last frame in the first series of MR image frames and the second representative image is a last frame in the second series of MR image frames.

In another embodiment, the first representative image is one of a last few frames in the first series of MR image frames and the second representative image is one of a last few frames in the second series of MR image frames.

In another embodiment, the first representative image includes multiple frames in the first series of MR image frames and the second representative image includes multiple frames in the second series of MR image frames.

In another embodiment, each of the first series of MR image frames and the second series of MR images frames is an inversion recovery series, and each of the first quantitative parameter map and the second quantitative parameter map is a relaxation map generated using an inversion recovery parameter fit. The first representative image and the second representative image are each obtained in the respective inversion recovery series after a magnetization of a tissue of the patient's anatomy returns to an equilibrium state.

In one embodiment, the patient's anatomy is the patient's heart and the comparison map is an extracellular volume (ECV) map.

In one embodiment, the first series of MR image frames is obtained before injection of a contrast agent and the second series of MR image frames is obtained after injection of the contrast agent. Optionally, the first quantitative parameter map and the second quantitative parameter map are either T1 maps or T2 maps.

In one embodiment, the first time period and the second time period are at least 5 minutes apart.

In another embodiment, the first time period and the second time period are at least 10 minutes apart.

In another embodiment, the first time period and the second time period are at least 15 minutes apart.

In another embodiment, the first time period and the second time period are more than one hour apart.

In another embodiment, each of the first series of MR image frames are motion compensated. Optionally, each of the second series of MR image frames are also motion compensated.

In another aspect of the disclosure, a system for processing acquired magnetic resonance MR images includes a processor and a memory comprising instructions configured to cause the processor to obtain a first series of MR image frames of a patient's anatomy over a first time period and a first quantitative parameter map based on the first series of MR image frames, and obtain a second series of MR image frames of the patient's anatomy over a second time period and a second quantitative parameter map based on the second series of MR image frames, wherein the first time period does not overlap with the second time period. The processor executing the instructions selects at least one MR image frame from the first series of MR image frames as a first representative image and selects at least one MR image frame from the second series of MR image frames as a second representative image, and then calculates a misalignment valuation based on a comparison of the first representative image to the second representative image. The processor executing the instructions compares the first quantitative parameter map and the second quantitative map based on the misalignment valuation to generate a comparison map.

In one embodiment, the first representative image is one of a last few frames in the first series of MR image frames and the second representative image is one of a last few frames in the second series of MR image frames.

In another embodiment, the first representative image includes multiple frames in the first series of MR image frames and the second representative image includes multiple frames in the second series of MR image frames.

In one embodiment, wherein each of the first series of MR image frames and the second series of MR images frames is an inversion recovery series and each of the first quantitative parameter map and the second quantitative parameter map is a relaxation map generated using an inversion recovery parameter fit, the first representative image and the second representative image are each obtained in the respective inversion recovery series after a magnetization of a tissue of the patient's anatomy returns to an equilibrium state.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a schematic diagram of an MRI system, in accordance with an exemplary embodiment;

FIG. 2 illustrates exemplary pre-contrast and post-contrast T1 maps.

FIG. 3 is a logic diagram illustrating one embodiment of motion correction for aligning and comparing quantitative maps in accordance with the present disclosure.

FIG. 4 is a flow chart illustrating one embodiment of a method of processing MR images in accordance with the present disclosure.

FIG. 5 is a flow chart illustrating another embodiment of a method of processing MR images in accordance with the present disclosure.

FIG. 6 exemplifies representative MR image frames selected for misalignment valuation for aligning T1 maps in accordance with an embodiment of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The inventors have recognized a need for tools to better align MRI maps generated based on MRI image sets taken a different times, such as for alignment of T1 maps or T2 maps generated based on images taken from a patient in two different imaging sessions that are several minutes, hours, or even days/weeks/months apart. Two MRI maps need to be aligned precisely so that a pixel-by-pixel comparison can be conducted.

In order to generate quantitative comparisons between MRI maps generated based on images taken from a patient at different time periods, significant image alignment adjustments are often needed. Major differences in patient position within the imager and changes in patient anatomy (such as due to the changed patient position) mean that the shapes and locations of anatomical aspects in the image will change. Image alignment is even more problematic where the maps being compared have significantly different intensities, such as comparing a map generated based on image frames generated without contrast agent to a map generated based on image frames generated with contrast agent injected into the patient. For example, several imaging techniques rely on a comparison of two relaxation maps (e.g., T1 maps or T2 maps), such as extracellular volume (ECV) mapping for measuring heart tissue to diagnose and/or assess certain heart conditions. ECV mapping compares T1 maps taken from a patient prior to injection of a contrast agent and after injection of a contrast agent and requires precise alignment of the pre-contrast and post-contrast T1 maps.

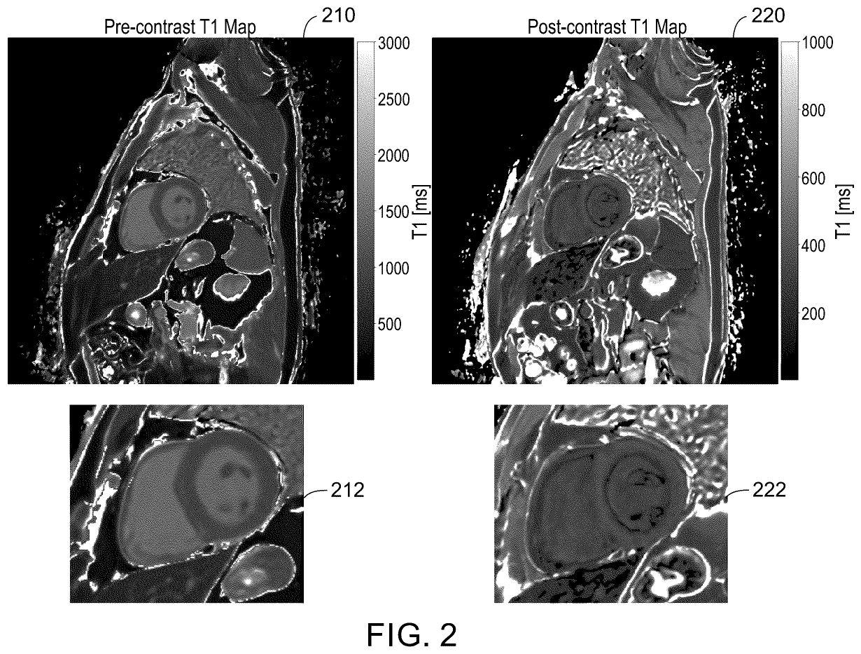

This problem of alignment is a long-standing problem, particularly in ECV mapping and similar comparison mapping of pre-contrast and post-contrast T1 or T2 maps. Existing image alignment techniques, such as intensity-based alignment algorithms, segmentation-based alignment algorithms, and edge-detection-based alignment algorithms have high failure rate when comparing maps based on images taken at different times and/or when comparing maps generated based on images with and without contrast. Such differences are exemplified in FIG. 2, which shows exemplary pre-contrast and post-contrast T1 maps. T1 map 210 was generated based on a first series of MR images taken without a contrast agent and T2 map 220 was generated based on a second series of MR images taken with a contrast agent. As can be seen by comparing these T1 maps 210 and 220, the pixel intensity values between the two images are very different. Further, the anatomical features have different shapes and locations, partly due to the differences in cellular behavior due to difference caused by the contrast agent and also due to changes in patient position and patient location in the imager when the pre-contrast and post-contrast image series were taken. There is typically at least 15 minutes between pre-contrast and post-contrast images, and during that time the patient is likely to shift position.

Due to these differences in pre-contrast and post-contrast T1 maps, standard image alignment techniques, such as those listed above, are inconsistent and cannot be relied upon for producing reliable clinical assessments. Thus, many existing software tools for generating comparison maps require manual input from a clinician to identify certain key anatomical structures, features, and/or boundaries. For example, existing ECV mapping software and tools require a clinician to outline the portion of each of the T1 maps associated with the patient's heart. This requires clinician time and also introduces inaccuracy due to human error.

In view of the forgoing long-standing problem in the relevant art and challenges recognized by the inventors, the disclosed method and system were developed wherein a misalignment valuation is conducted using the image frames that the quantitative parameter maps (e.g., the pre-contrast and post-contrast T1 maps) are based on. The misalignment calculation is not based on the quantitative parameter maps (e.g., the T1 maps), but is based on a comparison of representative image frames from each of the image series used to generate the maps. The misalignment valuation is then applied to the maps to align them. Thus, the inventors have recognized that determining a misalignment between a representative image frame from each of the image series is sufficiently representative of the misalignment between the quantitative parameter maps. In particular, the inventors have recognized that the last image frame in each image series, or otherwise at least one image frame towards the end of the series, have similar intensity levels because the spins are back to their equilibrium state after the inversion. Thus, the first representative image and the second representative images are taken from the end portion of their respective inversion recovery series after a magnetization of a tissue of the patient's anatomy returns to an equilibrium state. Comparing the image frames from the end portion of each inversion recovery series provides particularly good alignment valuation that is sufficiently similar to and sufficiently represents the misalignment between the T1 maps (or T2 maps) generated based on the image series. For example, the misalignment valuation may be conducted using an intensity-based motion correction algorithm, examples of which are well known in the art.

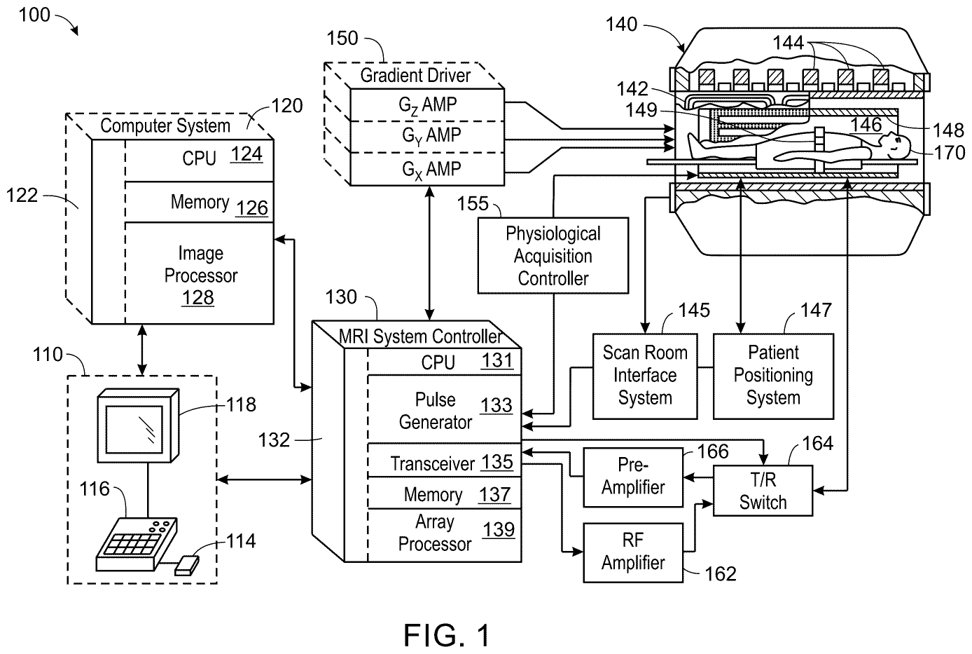

Referring to FIG. 1, a schematic diagram of an exemplary MRI system 100 is shown in accordance with an embodiment. The operation of MRI system 100 is controlled from an operator workstation 110 that includes an input device 114, a control panel 116, and a display 118. The input device 114 may be a joystick, keyboard, mouse, track ball, touch activated screen, voice control, or any similar or equivalent input device. The control panel 116 may include a keyboard, touch activated screen, voice control, buttons, sliders, or any similar or equivalent control device. The operator workstation 110 is coupled to and communicates with a computer system 120 that enables an operator to control the production and viewing of images on display 118. The computer system 120 includes a plurality of components that communicate with each other via electrical and/or data connections 122. The computer system connections 122 may be direct wired connections, fiber optic connections, wireless communication links, or the like. The components of the computer system 120 include a central processing unit (CPU) 124, a memory 126, which may include a frame buffer for storing image data, and an image processor 128. In an alternative embodiment, the image processor 128 may be replaced by image processing functionality implemented in the CPU 124. The computer system 120 may be connected to archival media devices, permanent or back-up memory storage, or a network. The computer system 120 is coupled to and communicates with a separate MRI system controller 130.

The MRI system controller 130 includes a set of components in communication with each other via electrical and/or data connections 132. The MRI system controller connections 132 may be direct wired connections, fiber optic connections, wireless communication links, or the like. The components of the MRI system controller 130 include a CPU 131, a pulse generator 133, which is coupled to and communicates with the operator workstation 110, a transceiver 135, a memory 137, and an array processor 139. In an alternative embodiment, the pulse generator 133 may be integrated into a resonance assembly 140 of the MRI system 100. The MRI system controller 130 is coupled to and receives commands from the operator workstation 110 to indicate the MRI scan sequence to be performed during an MRI scan. The MRI system controller 130 is also coupled to and communicates with a gradient driver system 150, which is coupled to a gradient coil assembly 142 to produce magnetic field gradients during an MRI scan.

The pulse generator 133 may also receive data from a physiological acquisition controller 155 that receives signals from a plurality of different sensors connected to an object or patient 170 undergoing an MRI scan, including electrocardiography (ECG) signals from electrodes attached to the patient 170. And finally, the pulse generator 133 is coupled to and communicates with a scan room interface system 145, which receives signals from various sensors associated with the condition of the resonance assembly 140. The scan room interface system 145 is also coupled to and communicates with a patient positioning system 147, which sends and receives signals to control movement of a table 171. The able 171 is controllable to move the patient in and out of the core 146 and to move the patient to a desired position within the core 146 for an MRI scan.

The MRI system controller 130 provides gradient waveforms to the gradient driver system 150, which includes, among others, G_{X}, G_{Y} and G_{Z} amplifiers. Each G_{X}, G_{Y} and G_{Z} gradient amplifier excites a corresponding gradient coil in the gradient coil assembly 142 to produce magnetic field gradients used for spatially encoding MR signals during an MRI scan. The gradient coil assembly 142 is included within the resonance assembly 140, which also includes a superconducting magnet having superconducting coils 144, which in operation, provides a homogenous longitudinal magnetic field B₀ throughout a core 146, or open cylindrical imaging volume, that is enclosed by the resonance assembly 140. The resonance assembly 140 also includes a RF body coil 148 which in operation, provides a transverse magnetic field B₁ that is generally perpendicular to B₀ throughout the core 146. The resonance assembly 140 may also include RF surface coils 149 used for imaging different anatomies of a patient undergoing an MRI scan. The RF body coil 148 and RF surface coils 149 may be configured to operate in a transmit and receive mode, transmit mode, or receive mode.

An object or patient 170 undergoing an MRI scan may be positioned within the core 146 of the resonance assembly 140. The transceiver 135 in the MRI system controller 130 produces RF excitation pulses that are amplified by an RF amplifier 162 and provided to the RF body coil 148 and RF surface coils 149 through a transmit/receive switch (T/R switch) 164.

As mentioned above, RF body coil 148 and RF surface coils 149 may be used to transmit RF excitation pulses and/or to receive resulting MR signals from a patient undergoing an MRI scan. The resulting MR signals emitted by excited nuclei in the patient undergoing an MRI scan may be sensed and received by the RF body coil 148 or RF surface coils 149 and sent back through the T/R switch 164 to a pre-amplifier 166. The amplified MR signals are demodulated, filtered and digitized in the receiver section of the transceiver 135. The T/R switch 164 is controlled by a signal from the pulse generator 133 to electrically connect the RF amplifier 162 to the RF body coil 148 during the transmit mode and connect the pre-amplifier 166 to the RF body coil 148 during the receive mode. The T/R switch 164 may also enable RF surface coils 149 to be used in either the transmit mode or receive mode.

The resulting MR signals sensed and received by the RF body coil 148 are digitized by the transceiver 135 and transferred to the memory 137 in the MRI system controller 130.

A MR scan is complete when an array of raw k-space data, corresponding to the received MR signals, has been acquired and stored temporarily in the memory 137 until the data is subsequently transformed to create images. This raw k-space data is rearranged into separate k-space data arrays for each image to be reconstructed, and each of these separate k-space data arrays is input to the array processor 139, which operates to Fourier transform the data into arrays of image data.

The array processor 139 uses a known transformation method, most commonly a Fourier transform, to create images from the received MR signals. These images are communicated to the computer system 120 where they are stored in memory 126. In response to commands received from the operator workstation 110, the image data may be archived in long-term storage or it may be further processed by the image processor 128 and conveyed to the operator workstation 110 for presentation on the display 118.

In various embodiments, the components of computer system 120 and MRI system controller 130 may be implemented on the same computer system or a plurality of computer systems. Similarly, each of the computer system 120 and MRI system controller 130 may be implemented using a single processor, or multiple processors networked together or otherwise communicatively connected.

The imaging system, such as by the MRI system controller 130, may be controlled to perform an magnetization recovery series of images such that the system 100 generates a relaxation map. Relaxation mapping in MRI refers to a technique used to create a map of tissue T1 relaxation times or a map of T2 relaxation times by utilizing an pulse sequence, which involves applying a preparation radiofrequency pulse to flip the magnetization of the tissue before acquiring the signal, allowing for the measurement of how quickly the magnetization returns to its equilibrium state at different tissue types, thus generating a “T1 map” or a or “T2 map” of the scanned region. A T1 map indicates a tissue's “longitudinal relaxation time” (T1), while a T2 map indicates a tissue's “transverse relaxation time” (T2).

The critical parameter in T1 mapping is the “inversion time” (TI), which is the time interval between the 180° preparation pulse and the excitation pulse for signal acquisition. An alternative approach uses 90° saturation preparation pulses with a time interval “saturation time” (TS). By acquiring multiple images with different TIs, a relaxation time can be calculated for each pixel in the image, creating a T1 map that can be used to differentiate tissues based on their relaxation properties. Relaxation mapping is particularly useful in assessing tissue pathology in the brain, heart, and musculoskeletal system, as changes in tissue composition can significantly alter relaxation times. A widely used method for T1 mapping, especially in cardiac imaging, is the Modified Look-Locker Inversion Recovery (MOLLI) imaging method where multiple inversion pulses are applied with varying TIs to efficiently acquire a series of MR image frames and generate a T1 map based thereon.

A quantitative parameter map is then generated based on the series of MR image frames captured. For example, the quantitative parameter map may be a relaxation map, such as a T1 map or a T2 map, generated using a magnetization recovery parameter fit. A magnetization recovery parameter fit refers to the process of mathematically analyzing data acquired using a magnetization recovery (e.g., inversion recovery (IR)) MRI sequence to extract tissue properties (e.g., the longitudinal relaxation time (T1)), by fitting the measured signal intensities to a function that describes the signal recovery after a preparation pulse. This allows for quantitative mapping across different tissues within an image.

FIG. 2 shows exemplary quantitative parameter maps, which here are T1 maps. In other embodiments, a different type of relaxation map may be generated, such as a T2 map. T1 map 210 is generated based on a first series of MR image frames taken without a contrast agent and T2 map 220 is generated based on a second series of MR image frames taken with a contrast agent. The T1 maps 212 and 222 show portions of the respective T1 maps 210 and 220 containing the heart, which is the target anatomy to be imaged. The T1 map images 210, 212, 220, 222 shown in FIG. 2 are grey-scale images; however, a person of ordinary skill in the art will recognize that T1 images (and T2 images and other map types) are typically in color and that different color schemes may be used to represent the image scale.

As can be seen by comparing these T1 maps 210 and 220, the pixel intensity values between the two images are very different. The pixel-by-pixel differences between the T1 maps 210 and 220 (or just between the map portions 212 and 222 containing the heart) may be quantified in a comparison map. The comparison map describes the differences in tissue behavior between the map generated without contrast and the map generated with contrast. One exemplary comparison map is an extracellular volume (ECV) map, which is a cardiac magnetic resonance image that estimates the amount of space in the heart's extracellular compartments. ECV mapping is a noninvasive technique that can help diagnose and predict the course of heart conditions.

ECV mapping is performed by taking a series of MR image frames before and after a contrast agent is injected. For ECV, a gadolinium-based contrast agent is injected into the patient. T1 maps of the patient's T1 values before and after the contrast agent is injected are calculated based on the before and after series of MR image frames. The change in T1 values is used to calculate the ECV, and an ECV map is generated. Generating the ECV map requires a pixel-by-pixel alignment of the pre-contrast and post-contrast T1 maps 210 and 220 (or at least map portions 212 and 222).

Thus, the pre-contrast and post-contrast T1 maps 210 and 220 (or at least map portions 212 and 222) must be accurately aligned so that a pixel-by-pixel comparison can be made to generate the ECV map. The disclosed system and method is configured to calculate an alignment valuation that can be used to align the pre-contrast and post-contrast T1 maps. The alignment valuation is calculated based on a comparison between one representative image frame from the pre-contrast image series used to generate the pre-contrast T1 map and one representative image frame from the post-contrast image series used to generate the post-contrast T1 map. The ECV map is then generated by applying the alignment valuation to the T1 maps.

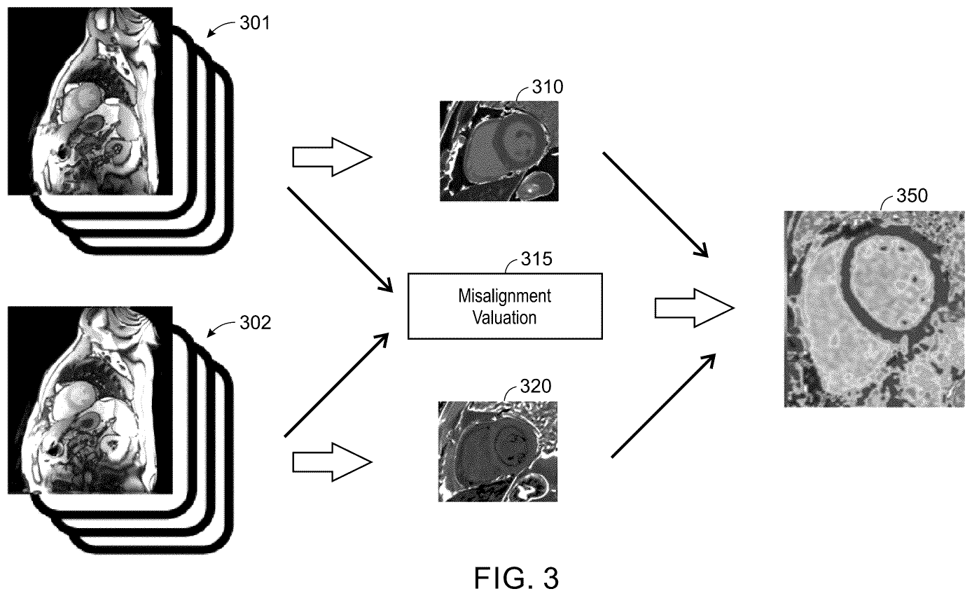

FIG. 3 represents an exemplary workflow for generating a comparison map, such as an ECV map, using the disclosed method and system. A first series of MR image frames 301 is obtained via an MRI system over a first time period. For example, a MOLLI imaging protocol may be employed to generate each series of MR image frames. For example, the MOLLI protocol may employ two inversions to acquire a predetermined number of image frames (e.g., eight image frames) over a predetermined number of heartbeats (e.g., 11 heartbeats). In one example, five image frames are acquired over consecutive cardiac cycles followed by a three heartbeat gap and then three image frames are acquired over consecutive cardiac cycles. Such MOLLI protocol would acquire images for an (approximate) duration of 5 s followed by a gap of 3 s and a second acquisition train lasting 3 s.

The first series of MR image frames 301 is an inversion recovery series taken without contrast agent. Then a contrast agent is injected into the patient. After a waiting period for absorption of the contrast agent, a second series of MR image frames 302 is obtained using the MRI system over a second time period. In one embodiment, the time between the first time period of the first image series and the second time period of the second image series is at least 10 minutes, and in many embodiments is at least 15 minutes. However, in other implementations, the time period between the first and second image series may be greater than 15 minutes or may be less than 10 minutes, which may depend on the contrast agent, the anatomy being imaged, and/or certain patient-specific parameters.

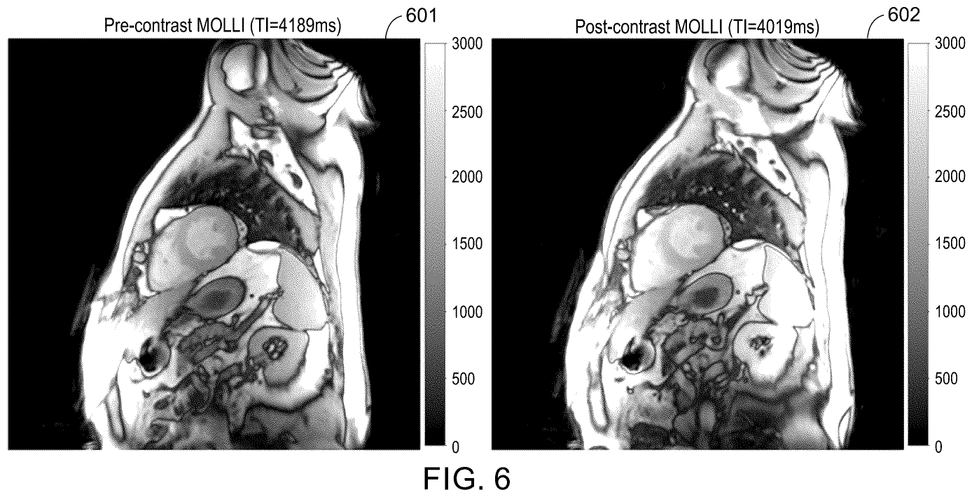

The misalignment valuation 315 is calculated based on representative images from each of the first series of MR image frames 301 and the second series of MR image frames 302. The representative image frame is preferably selected to minimize the differences in intensity values between the two images. For example, where each of the series of MR image frames is an inversion recovery series, the representative image may be taken from the end portion of each respective inversion recovery series after a magnetization of a tissue of the patient's anatomy has returned to an equilibrium state. (See FIG. 6 and the corresponding description below.) In one embodiment, the first representative image is a last frame in the first series of MR image frames and the second representative image is a last frame in the second series of MR image frames. In other embodiments, the last few image frames may be sufficiently representative. For example, the first representative image may be one or more of the last two frames in the first series of MR image frames and the second representative image may be one of the last two frames in the second series of MR image frames. In some implementations, the representative image may include multiple frames from the respective series of MR image frames. For example, the representative image may be an average of two or more image frames or some other calculation based on multiple frames. Optionally, each of the first series of MR image frames and/or the second MR image frames are motion compensated. Thus, the representative images may include one or more motion compensated image frames from the respective series.

In one embodiment, the misalignment valuation 315 is a map of pixel-by-pixel alignment values (e.g., a vector field) representing the motion between the first representative frame and the second representative frame. In another embodiment, the misalignment valuation 315 is a set of values representing a subset of pixel alignments between the first and second representative frames, and interpolation (e.g., by B-spline functions) is used to align the remaining pixels. In still other embodiments, the misalignment valuation 315 may be described as a parametric correspondence relationship (e.g., using a model of cardiac or cardiorespiratory motion), or may be a feature-based alignment output. The format of the misalignment valuation 315 will depend on the alignment algorithm used. In one embodiment, the alignment algorithm is an intensity-based alignment algorithm (e.g., based on a mean of squared pixel-wise differences, a cross-correlation, or a mutual information metric). Since the representative images are chosen to have similar intensity values, an intensity-based comparison may be appropriate. In other embodiments, a segmentation-based (e.g., based on artificial intelligence cardiac segmentation models) or an edge-detection-based (e.g., Canny edge detector) alignment algorithm may be utilized, or any image alignment algorithm suited for aligning 2D images (particularly grey-scale MRI images).

A quantitative parameter map is then generated for each series of MR image frames. A first quantitative parameter map 310 is generated based on the first series of MR image frames (the pre-contrast image frames) and a second quantitative parameter map 320 is generated based on the second series of MR image frames (the post-contrast image frames). Here, the first and second quantitative parameter maps are T1 maps. The T1 map images 310 and 320 shown in FIG. 3 are grey-scale images; however, a person of ordinary skill in the art will recognize that T1 images (and T2 images and other map types) are typically color images and that different color schemes may be used to indicate the value scale represented in the image.

The alignment information is then used to align the first and second quantitative parameter maps 310 and 320 to generate a comparison map 350. The alignment information is used to align the pixels of the first and second quantitative parameter maps 310 and 320 to one another. The comparison map provides information about the differences between the first and second quantitative parameter maps 310 and 320. For example, the comparison map may be a pixel-by-pixel comparison, such as pixel intensity comparison, between the first and second quantitative parameter maps 310 and 320. The comparison map 350 shown in FIG. 3 is an ECV map. ECV mapping technique and applications therefor are described in detail above. The ECV map image 350 shown in FIG. 3 is a grey-scale image; however, a person of ordinary skill in the art will recognize that ECV images (and other comparison image types) are typically in color and that different color schemes may be used to indicate the value scale represented in the image.

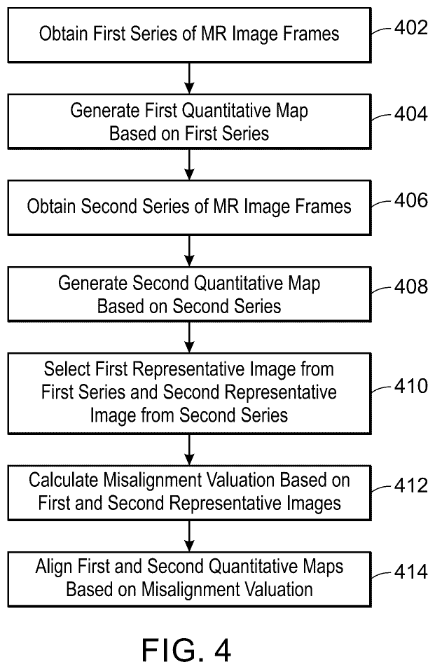

FIG. 4 is a flow chart illustrating one embodiment of a method of processing MR images. A first series of MR image frames are obtained at step 402, which were generated and stored by the MRI system. For example, the MR image frames may be an inversion recovery series, as described above. A first quantitative map, such as a T1 map or a T2 map, is then generated at step 404 based on the first series of MR image frames. A second series of MR image frames is obtained at step 406, which were generated and stored by the MRI system at a different time than the first series. The second series of MR image frames may be the same type of image series as the first series of MR image frames, but taken at a later time. For example, the second series of MR image frames may be taken at least 10 minutes after the first series, such as after delivery of a contrast agent or execution of a procedure. In other examples, the second series may be hours or even days later. A second quantitative map, such as a T1 map or a T2 map, is then generated at step 408 based on the second series of MR image frames.

A representative image from each series is then selected at step 410. Namely, a first representative image is selected from the first series of MR image frames and a second representative image is selected from the second series of MR image frames. For example, the representative images may be chosen to have similar intensity values, such as images representing an equilibrium state of the magnetization of the tissues in the image.

A misalignment valuation is then calculated at step 412. For example, an intensity-based alignment algorithm may be utilized to generate alignment information that characterizes the distances between corresponding pixels in each of the first and second representative images. In other embodiments, a different type of alignment algorithm may be used, as is described above. The alignment valuation is then utilized to align the first and second quantitative maps at step 414. For example, the aligned first and second quantitative maps may be compared to generate a comparison that quantifies the differences therebetween.

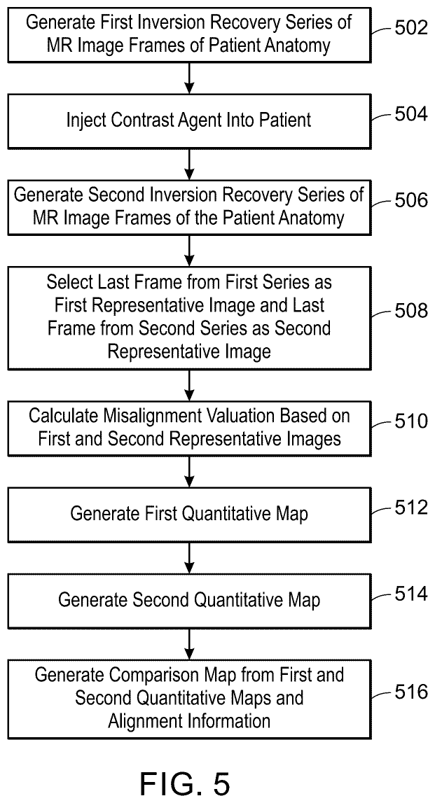

FIG. 5 is another flow chart illustrating an embodiment of a method of processing MR images. An MRI system is controlled to generate and store a first inversion recovery series of MR image frames of a patient's anatomy at step 502. A contrast agent is then injected into the patient at step 504. In some implementations, the patient is moved out of the bore after the first series of MR frames is obtained and the contrast agent is then administered and a wait time is facilitated to enable uptake of the contrast agent. Regardless of whether the patient is moved out of the bore, they are likely to shift position on the table and to otherwise move their body during the wait time, which is at least several minutes and typically around fifteen minutes. After the wait time, the patient is moved back into the bore (if they were moved out) and the MRI system is controlled to generate and store a second inversion recovery series of MR image frames of the patient's anatomy at step 506.

A representative image is then selected from each of the first series of MR image frames and the second series of MR image frames, represented as step 508. In the depicted embodiment, the last frame of each series of MR image frames is selected as the representative frame to maximize the inversion time and thus the likelihood that the magnetization of the tissues in the imaged anatomy have returned to their equilibrium state after the inversion. As described above, this increases the likelihood that the represented images of the inversion recovery series will have sufficiently similar intensity levels. FIG. 6 illustrates this concept, showing exemplary representative images 601 and 602 that are the last images in first and second series of image frames. The first representative image 601 is the last MR image frame in a first series of MOLLI images taken before delivery of a contrast agent, which is taken at an inversion time (TI) of 4.189 seconds. The second representative image 602 is the last MR image frame in a second series of MOLLI images taken after delivery of the contrast agent, which has a TI of 4.019 seconds. The first representative image 601 and second representative image 602 have more similar intensity levels than do the image frames at the beginning and middle of the first and second image series. Likewise, the first representative image 601 and second representative image 602 have more similar intensity levels than the T1 maps or other parameter maps generated from the image series. Thus, the misalignment valuation between the representative images 601 and 602 is more likely to be accurate than the same misalignment calculation between the quantitative maps. The more accurate misalignment valuation can then be applied to align the maps.

A misalignment valuation between the first and second representative images is calculated at step 510, such as using an intensity-based alignment algorithm or any other appropriate alignment algorithms, examples and requirements for which are described herein. Quantitative maps are generated based on the image frames, such as T1 maps or T2 maps. Here, a first quantitative map is generated at step 512 based on the pre-contrast inversion recovery series of MR image frames, and a second quantitative map is generated at step 514 based on the post-contrast inversion recovery series of MR image frames. Quantitative mapping may be performed before, after, or substantially simultaneously with calculation of the misalignment valuation.

The alignment valuation is then utilized to align the first and second quantitative maps. The aligned maps are then compared to generate a comparison map at step 516. Thus, even though the alignment valuation was conducted using just two individual image frames, rather than the quantitative maps, the alignment valuation is a sufficiently accurate description of the misalignment between the two quantitative maps. The pixel locations in the representative images 601 and 602 are not identical to the quantitative maps calculated based on the respective image series. However, the inventors have recognized that the representative images are close enough to the resulting maps that the misalignment valuation based on the representative image frames provides sufficient accuracy for aligning the quantitative maps to generate comparison maps. Furthermore, the results of the alignment of the representative images may optionally be used as initialization for a second alignment stage based on the quantitative maps.

It should be understood that the above-described steps of the processes of FIGS. 4 and 5 can be executed or performed in any suitable order or sequence not limited to the order and sequence shown and described in the figures. Also, some of the above steps of the processes of FIGS. 4 to 5 can be executed or performed substantially simultaneously where appropriate or in parallel to reduce latency and processing times.

It should be noted that, as used herein, the term mechanism can encompass hardware, software, firmware, or any suitable combination thereof.

It should be understood that the image frames can be reconstructed from fully sampled or undersampled k-space data. Undersampled k-space data can be reconstructed with parallel imaging techniques (e.g. SENSE, GRAPPA and ARC), compressed sensing (CS), or deep learning (e.g. Sonic DL). The image reconstruction technique can utilize temporal correlations between frames (e.g. using k-t SENSE, k-t GRAPPA, k-t ARC, k-t CS, or k-t Sonic DL). Data sampling and reconstruction can use Cartesian sampling, or other type of sampling strategies (e.g. spiral, radial).

In various embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

This written description uses examples to disclose the invention(s), including the best mode, and also to enable any person skilled in the art to make and use the invention(s). Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention(s) is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A method of processing magnetic resonance (MR) images, the method comprising:
obtaining a first series of MR image frames of a patient's anatomy over a first time period;
generating a first quantitative parameter map based on the first series of MR image frames;
obtaining a second series of MR image frames of the patient's anatomy over a second time period, wherein the first time period does not overlap with the second time period;
generating a second quantitative parameter map based on the second series of MR image frames;
selecting at least one MR image frame from the first series of MR image frames as a first representative image;
selecting at least one MR image frame from the second series of MR image frames as a second representative image;
calculating a misalignment valuation based on a comparison of the first representative image to the second representative image; and
comparing the first quantitative parameter map and the second quantitative map based on the misalignment valuation to generate a comparison map.

2. The method of claim 1, wherein the first representative image is a last frame in the first series of MR image frames and the second representative image is a last frame in the second series of MR image frames.

3. The method of claim 1, wherein the first representative image is one of a last two frames in the first series of MR image frames and the second representative image is one of a last two frames in the second series of MR image frames.

4. The method of claim 1, wherein the first representative image includes multiple MR image frames in the first series of MR image frames and the second representative image includes multiple MR image frames in the second series of MR image frames.

5. The method of claim 1, wherein each of the first series of MR image frames and the second series of MR images frames is an inversion recovery series, and wherein each of the first quantitative parameter map and the second quantitative parameter map is a relaxation map generated using an inversion recovery parameter fit.

6. The method of claim 5, wherein the first representative image and the second representative image are each obtained in the respective inversion recovery series after a magnetization of a tissue of the patient's anatomy returns to an equilibrium state.

7. The method of claim 5, wherein the patient's anatomy is the patient's heart and the comparison map is an extracellular volume (ECV) map.

8. The method of claim 5, wherein the first series of MR image frames is obtained before injection of a contrast agent and the second series of MR image frames is obtained after injection of the contrast agent.

9. The method of claim 5, wherein the first quantitative parameter map and the second quantitative parameter map are either T1 maps or T2 maps.

10. The method of claim 1, wherein each of the first series of MR image frames are motion compensated.

11. The method of claim 10, wherein each of the second series of MR image frames are motion compensated.

12. The method of claim 1, wherein the first series of MR image frames is obtained before injection of a contrast agent and the second series of MR image frames is obtained after injection of the contrast agent.

13. The method of claim 1, wherein the first time period and the second time period are at least 10 minutes apart.

14. A system for processing acquired magnetic resonance MR images, the system comprising:
a processor; and
a memory comprising instructions configured to cause the processor to:
obtain a first series of MR image frames of a patient's anatomy over a first time period and a first quantitative parameter map based on the first series of MR image frames;
obtain a second series of MR image frames of the patient's anatomy over a second time period and a second quantitative parameter map based on the second series of MR image frames;
wherein the first time period does not overlap with the second time period;
select at least one MR image frame from the first series of MR image frames as a first representative image;
select at least one MR image frame from the second series of MR image frames as a second representative image;
calculate a misalignment valuation based on a comparison of the first representative image to the second representative image; and
compare the first quantitative parameter map and the second quantitative map based on the misalignment valuation to generate a comparison map.

15. The system of claim 14, wherein the first representative image is a last frame in the first series of MR image frames and the second representative image is a last frame in the second series of MR image frames.

16. The system of claim 14, wherein the first representative image includes multiple MR image frames in the first series of MR image frames and the second representative image includes multiple MR image frames in the second series of MR image frames.

17. The system of claim 14, wherein each of the first series of MR image frames and the second series of MR images frames is an inversion recovery series, and wherein each of the first quantitative parameter map and the second quantitative parameter map is a relaxation map generated using an inversion recovery parameter fit.

18. The system of claim 17, wherein the first representative image and the second representative image are each obtained in the respective inversion recovery series after a magnetization of a tissue of the patient's anatomy returns to an equilibrium state.

19. The system of claim 17, wherein the patient's anatomy is the patient's heart and the comparison map is an extracellular volume (ECV) map.

20. The system of claim 17, wherein the first series of MR image frames is obtained before injection of a contrast agent and the second series of MR image frames is obtained after injection of the contrast agent.

21. The system of claim 17, wherein the first quantitative parameter map and the second quantitative parameter map are either T1 maps or T2 maps.

22. The system of claim 17, wherein the first series of MR image frames is obtained before injection of a contrast agent and the second series of MR image frames is obtained after injection of the contrast agent.

23. The system of claim 17, wherein the first time period and the second time period are at least 5 minutes apart.
